(19) United States
(12) Patent Application Publication
(10) Pub. No.: US 2002/0115973 A1
(43) Pub. Date: Aug. 22, 2002

(54) Cotton ball having resilient net or web covering

(76) Inventor: Su Jen Chou, Ho Mei Town (TW)

Correspondence Address: CHARLES E. BAXLEY, 59 John Street - 5th Floor, New York, NY, 10038, US

(21) Appl. No.: 09/784,281

(22) Filed: Feb. 16, 2001

(51) Int. Cl. A61F 13/15; A61F 13/20

(52) U.S. Cl. 604/384; 604/377

(57) ABSTRACT

A cotton ball includes a cotton material having an outer peripheral portion, and a net or web covering engaged onto the outer peripheral portion of the cotton material for forming the cotton ball and for stably retaining the cotton material within the net or web covering. The net or web covering includes a number of threads secured together. The threads may include a number of resilient threads and a number of cotton threads or fiber threads for adjusting the resilience of the net or web covering and for suitably reducing the resilience of the net or web covering.

### BACKGROUND OF THE INVENTION

1. Field of the Invention

The present invention relates to a cotton ball, and more particularly to a cotton ball having a resilient net or web covering for retaining the cotton yarns and/or the cotton fibers in place.

2. Description of the Prior Art

As shown in FIG. 8, the typical cotton balls 18 are made of or include a number of cotton yarns 19, cotton waddings, cotton fibers, etc., that are randomly secured together. The cotton yarns 19 may not be solidly retained together and may be attached onto the users when the cotton ball 18 is engaged onto the bloody wound.

The present invention has arisen to mitigate and/or obviate the afore-described disadvantages of the conventional cotton balls.

### SUMMARY OF THE INVENTION

The primary objective of the present invention is to provide a cotton ball including a resilient net or web covering for retaining the cotton yarns and/or the cotton fibers in place and for preventing the cotton yarns and/or the cotton fibers from being disengaged from the cotton balls and from being attached onto the users.

In accordance with one aspect of the invention, there is provided a cotton ball comprising a cotton material including an outer peripheral portion, and a net covering engaged onto the outer peripheral portion of the cotton material for forming the cotton ball and for stably retaining the cotton material within the net covering.

The net covering includes a plurality of threads secured together. The threads may include a plurality of resilient threads and a plurality of cotton threads or fiber threads for adjusting the resilience of the net covering and for suitably reducing the resilience of the net covering and for preventing the cotton material from being squeezed or compressed together to a tiny volume.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is a perspective view of a prototype of a cotton strip for making the cotton ball in accordance with the present invention;

FIG. 2 is an enlarged partial plane view of the cotton strip;

FIGS. 3, 4, 5, 6 are perspective views illustrating the formation of the cotton ball in accordance with the present invention;

FIG. 7 is a perspective view illustrating the application and the operation of the cotton ball; and

FIG. 8 is a perspective view illustrating the operation of the typical cotton ball.

### DETAILED DESCRIPTION OF THE PREFERRED EMBODIMENT

Referring to the drawings, and initially to FIGS. 1-6, a cotton ball in accordance with the present invention comprises a cotton material 31 made of such as the cotton, the synthetic fiber, the polyester fibers, or the like. The cotton material 31 may include a number of cotton yarns, cotton fibers, cotton threads, etc. disposed together and having an excellent flexibility. A net or web covering 32 is engaged on the outer peripheral portion of the cotton material 31 for forming a tubular cotton strip as shown in FIG. 1. The cotton yarns and the cotton fibers and the cotton threads preferably include the same or the similar length.

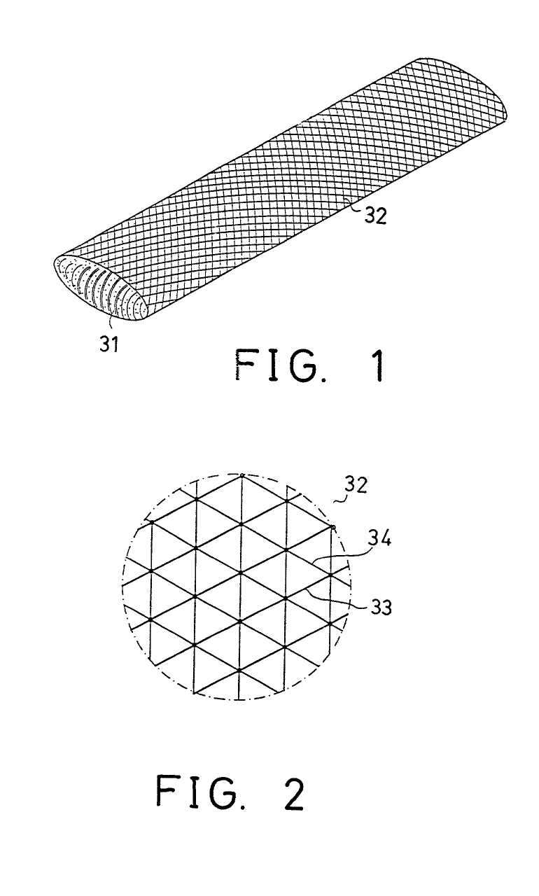

As shown in FIG. 2, the net or web covering 32 may include a number of fibers or threads 33, 34, woven together or coupled or secured together. For example, the net or web covering 32 may include a number of resilient threads 33 and the other fiber threads or cotton threads 34 or the like woven together. The cotton threads 34 may include a resilience less than that of the resilient threads 33 for reducing the resilience of the net or web covering 32 and for adjusting the resilience of the net or web covering 32 and for suitably reducing the resilience of the net or web covering 32 and for preventing the cotton material 31 from being squeezed or compressed together to a tiny volume. The squeezed or compressed cotton material 31 may not be used for suitable absorbing the fluid or the blood or the like.

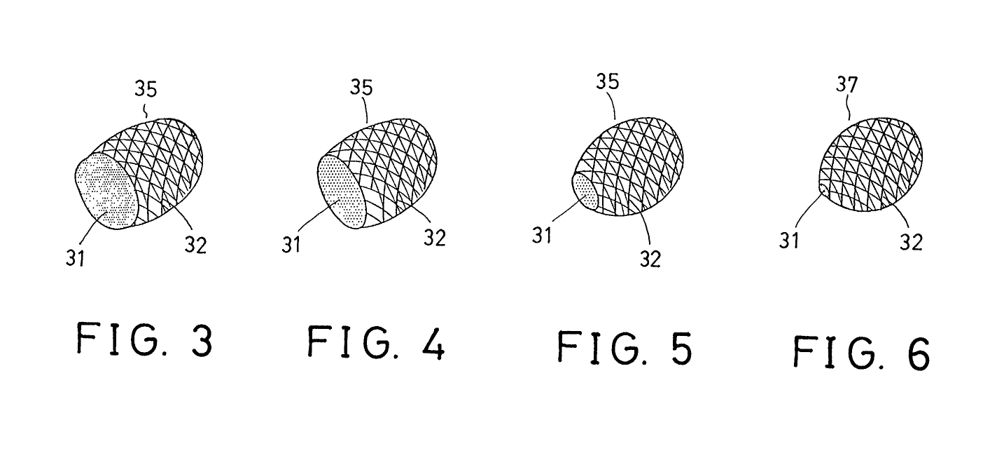

As shown in FIGS. 3-6, and again in FIG. 1, the tubular cotton strip as shown in FIG. 1 may then be cut into a number of segments 35 (FIG. 3). The resilience of the net or web covering 32 may then apply a resilient force against the cotton material 31 and may force the ends of the segments 35 toward or together to form the cotton ball 37 as shown in FIG. 6. The net or web covering 32 is also engaged on or provided on the outer peripheral portion of the cotton material 31 of the cotton ball 37. The cotton yarns and/or the cotton fibers and/or the cotton threads of the cotton material 31 thus may be solidly retained in the cotton ball 37 and will not be easily disengaged from the cotton ball 37.

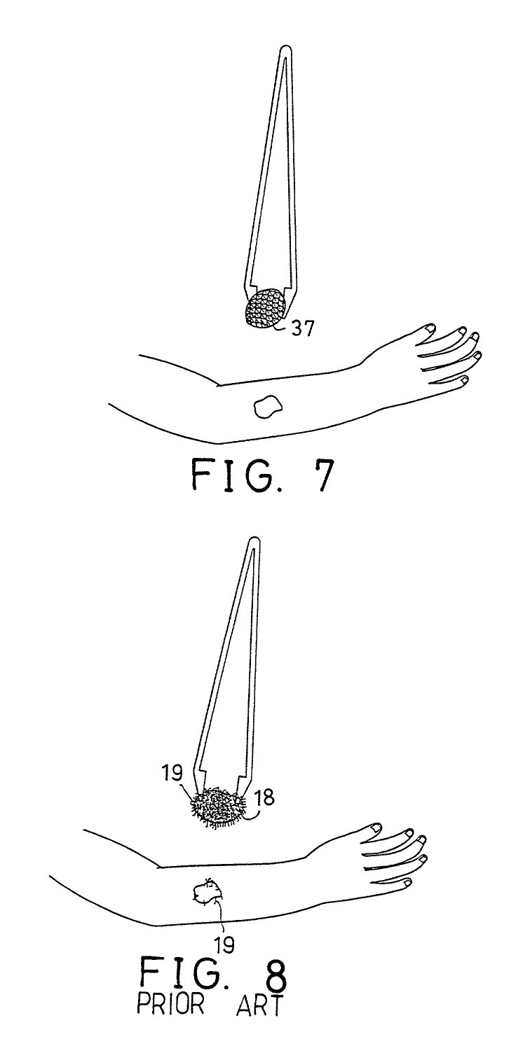

In operation, as shown in FIG. 7, the cotton yarns and/or the cotton fibers and/or the cotton threads of the cotton material 31 of the cotton ball 37 may thus be formed or retained as an integral element, such that the cotton ball 37 may be easily attached onto the users. The cotton yarns and/or the cotton fibers and/or the cotton threads of the cotton material 31 of the cotton ball 37 will not be easily disengaged from the cotton ball 37 and attached onto the users.

Accordingly, the cotton ball in accordance with the present invention includes a resilient net or web covering for retaining the cotton yarns and/or the cotton fibers in place and for preventing the cotton yarns and/or the cotton fibers from being disengaged from the cotton balls and from being attached onto the users.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A cotton ball comprising:
a cotton material including an outer peripheral portion, and
a net covering engaged onto said outer peripheral portion of said cotton material for forming said cotton ball and for stably retaining said cotton material within said net covering.

2. The cotton ball according to claim 1, wherein said net covering includes a plurality of threads secured together.

3. The cotton ball according to claim 2, wherein said threads includes a plurality of resilient threads and a plurality of cotton threads.

4. The cotton ball according to claim 2, wherein said threads includes a plurality of resilient threads and a plurality of fiber threads.
